(19) United States
(12) Patent Application Publication
(10) Pub. No.: US 2013/0172966 A1
(43) Pub. Date: Jul. 4, 2013

(54) SYSTEM FOR CONTROLLING THE TEMPERATURE OF A PERSON

(71) Applicants: Eliahu Arad, Tel-Aviv (IL); Ittai Shamir, Binyamina (IL)

(72) Inventors: Eliahu Arad, Tel-Aviv (IL); Ittai Shamir, Binyamina (IL)

(21) Appl. No.: 13/581,612

(22) PCT Filed: Mar. 28, 2011
(86) PCT No.: PCT/IB2011/051293

§ 371 Date: Dec. 19, 2012

Related U.S. Application Data
(60) Provisional application No. 61/318,397, filed on Mar. 29, 2010.

(51) Int. Cl. A61F 7/00

(52) U.S. Cl. CPC A61F 7/0085
USPC 607/105

(57) ABSTRACT

A system for affecting a temperature of a patient, the system includes: a nose catheter that is adapted to be inserted into a nose upper channel and to receive a fluid; and a temperature control and fluid supply unit that is adapted to supply the fluid and control a temperature of the fluid so as to affect a temperature of a brain of a patient when the nose catheter is inserted into the nose upper channel of a patient and receives the first fluid; wherein the temperature control and fluid supply unit comprises: a thermoelectric heat pump that is arranged to transfer heat from a cold side to a hot side; a fluid block arranged to be cooled by the cold side and to cool the fluid when the fluid passes through the fluid block; and at least one heat transfer element arranged to transfer heat from the hot side.

### CROSS REFERENCE TO RELATED APPLICATIONS

This application claims priority from U.S. provisional patent 61/318,397 filing date Mar. 29, 2010 which is incorporated in its entirety herein by reference.

### FIELD OF THE INVENTION

This disclosure relates generally to a system and method for manipulating a temperature of a patient.

### BACKGROUND OF THE INVENTION

Brain damage following stoke and after successful cardiac resuscitation remains a major cause of morbidity and mortality worldwide. Stroke is the third cause of death in developed countries, ranking only behind cancer and heart disease. Over the last several years animal studies have demonstrated some brain protection by inducing mild brain cooling (32-35° C.).

It has been shown that a reduction of the brain temperature may substantially reduce the ischemic damage of the brain. Clinical trails in different patient's populations resulted in conflicting results. In both adults and children that were treated with hypothermia following major traumatic brain injuries there was no evidence of benefit outcome at 6 and 12 months after the injury. On the other hand hypothermia therapy was found to be of benefit in adults and newborns with a hypoxic-ischemic (leak of oxygen and blood supply to the brain) brain injury.

The treating physician has to apply an effective hypothermic therapy as soon as possible, and minimize the neurological injury. There are several approaches to induce hypothermia both invasive and non-invasive. Most of the currently published clinical studies used non-invasive external cooling approach. One approach is to cool the entire body inducing whole body hypothermia. The major drawbacks of this approach are the 1.slow cooling rate due to the large volume of the body 2. The cumbersome of the large devices that were used 3. The possible deleterious systemic effects such as metabolic, cardiovascular, pulmonary, coagulation and immunologic complications. These effects may occur during the hypothermia period as well as during the re warming time. The increased risk of systemic complications when using whole body hypothermia may outweigh the brain protective benefits of such therapy.

Since the human brain weigh is only 2% of the total body weight and it receives 20% of the resting cardiac output it is not necessary to cool the entire body in order to achieve a reduction of the brain temperature. Presently brain cooling devices include surface cooling with ice-packs or cooling helmets and invasive approaches such as naso-phyrayngeal cooling and intra-carotid cold flushes. The cooling time while using these modalities is short (less than 1 h), however it provides more preferential cooling of the superficial areas of the brain that are in adjacent to the cooling device.

### SUMMARY OF THE PRESENT INVENTION

The present invention provides a method and a device as described in the accompanying claims. Specific embodiments of the invention are set forth in the dependent claims. These and other aspects of the invention will be apparent from and elucidated with reference to the embodiments described hereinafter.

According to an embodiment of the invention a system is provided for affecting a temperature of a patient. The system may include: a nose catheter that may be adapted to be inserted into a nose upper channel and to receive a fluid; and a temperature control and fluid supply unit that may be adapted to supply the fluid and control a temperature of the fluid so as to affect a temperature of a brain of a patient when the nose catheter is inserted into the nose upper channel of a patient and receives the first fluid; wherein the temperature control and fluid supply unit may include: a thermoelectric heat pump that may be arranged to transfer heat from a cold side to a hot side; a fluid block arranged to be cooled by the cold side and to cool the fluid when the fluid passes through the fluid block; and at least one heat transfer element arranged to transfer heat from the hot side.

The system may include an input element for receiving fluid from an infusion bag and an output element for inserting the fluid into the infusion bag.

The output element may be a hollow and sharp needle that may be arranged to puncture the infusion bag.

The nose catheter comprises an input tube, an output tube and a tip that are in fluid communication with each other; wherein the input tube may be arranged to receive the fluid and to allow the fluid to cool the tip and to exit through the output tube.

At least one of the input tube and the output tube may include a mixture of a metal powder and at least one other material.

The metal powder may be placed such as not to contact an organ of the patient once inserted in the nose upper canal of the patient.

The at least one tube may include an inner lumen that may include the metal powder and an outer lumen that surrounds the inner lumen.

The at least one of the input tube and the output tube may include a mixture of particles of a heat conducting material and of another material.

The system may include a controller and a temperature sensor, wherein the controller may be arranged to control the system based on a desired fluid temperature and a temperature measured by the temperature sensor.

The temperature sensor may be arranged to contact fluid within the system and sense the temperature of the fluid.

The temperature sensor may be arranged to sense a temperature of fluid without contacting the fluid.

The temperature sensor may be arranged to sense a temperature of an organ of a patient once inserted in the nose upper canal of the patient.

The system may include a controllable power source arranged to control power supplied to the thermoelectric heat pump.

The at least one heat transfer element comprise a fan and a heat sink.

The overall weight of the system may range between 1 and Kilograms.

The temperature control and fluid supply unit may be adapted to supply the fluid and control the temperature of the fluid so as to cool the brain of a patient when the nose catheter may be inserted into the nose upper channel of a patient and receives the first fluid.

The system may include a stomach catheter that may be adapted to be inserted into a stomach of the patient and to receive a second fluid; and wherein the temperature control and fluid supply unit may be further adapted to supply the second fluid and control a temperature of the second fluid so as to affect a temperature of at least one organ that differs from the brain of the patient when the stomach catheter may be inserted into the nose upper channel of the patient and receives the second fluid.

The temperature control and fluid supply unit may be adapted to control temperatures of the fluid and of the second fluid so that during at least one period of time, the temperature of the fluid differs from the temperature of the second fluid.

The temperature control and fluid supply unit may be adapted to control temperatures of the first and second fluids so that during at least one other period of time, the temperature of the first fluid substantially equals the temperature of the second fluid.

The temperature control and fluid supply unit may be adapted to control temperatures of the first and second fluids so that during a first period of time, the stomach catheter and the nasal catheter cool organs of the patient and during a second period of time that followed the first period of time the nasal catheter cools the brain of the patient while the stomach catheter warms at least one other organ of the patient.

The system may include a carotid scarf that may be adapted to be surround a neck of the patient and to receive a third fluid; and wherein the temperature control and fluid supply unit may be further adapted to supply the third fluid and control a temperature of the second fluid so as to affect a temperature the brain of the patient when the stomach catheter is inserted into the nose upper channel of the patient and receives the second fluid.

Any combination of the mentioned above features and, additionally or alternatively, components of the system may be provided.

According to an embodiment of the invention a method is provided for affecting a temperature of a patient. The method may include: inserting a nose catheter into a nose upper channel of a patient; supplying a fluid to the nose catheter, by a temperature control and fluid supply unit, so as to affect a temperature of a brain of a patient; wherein the temperature control and fluid supply unit may include: a thermoelectric heat pump that may be arranged to transfer heat from a cold side to a hot side; a fluid block arranged to be cooled by the cold side and to cool the fluid when the fluid passes through the fluid block; and at least one heat transfer element arranged to transfer heat from the hot side.

The method may include supplying the fluid to the nose catheter so as to cool the brain of the patient.

The method may include: inserting a temperature sensor to the patient; sensing a temperature an organ of the patent, by the temperature sensor; and determining the temperature of the fluid in response to temperature sensed by the temperature sensor.

The method further may include inserting a stomach catheter into a stomach of the patient; and supplying a second fluid to the stomach catheter so as to affect a temperature of at least one organ that differs from the brain of the patient.

The method may include controlling temperatures of the first and second fluids so that during at least one period of time, the temperature of the first fluid differs from the temperature of the second fluid.

The method may include controlling temperatures of the fluid and of the second fluid so that during at least one other period of time, the temperature of the fluid substantially equals the temperature of the second fluid.

The method may include controlling temperatures of the fluid and of the second fluid so that during a first period of time, the stomach catheter and the nasal catheter cool organs of the patient and during a second period of time that followed the first period of time the nasal catheter cools the brain of the patient while the stomach catheter warms at least one other organ of the patient.

The method may include placing a carotid scarf around a neck of the patient; and supplying a third fluid to the carotid scarf, by a temperature control and fluid supply unit, so as to affect a temperature of a brain of a patient.

The method may include inserting a stomach catheter into a stomach of the patient; and supplying a second fluid to the stomach catheter so as to affect a temperature of at least one organ that differs from the brain of the patient.

The method may include inserting a stainless steel tip of the nasal catheter into the nose upper channel, providing the stainless steel tip first fluid via an inlet and draining the first fluid from the stainless steel tip through an outlet.

The method may include cooling the fluid by the cold side and transferring heat from the hot side by the at least one heat transfer elements.

The method may include receiving the fluid from an infusion bag and retuning the fluid to the infusion bag via an output element.

The method wherein the output element may be a hollow and sharp needle that may be arranged to puncture the infusion bag.

The at least one of the input tube and the output tube may include a mixture of a metal powder and at least one other material.

The metal powder may be placed such as not to contact an organ of the patient once inserted in the nose upper canal of the patient.

The at least one tube may include an inner lumen that may include the metal powder and an outer lumen that surrounds the inner lumen.

The at least one of the input tube and the output tube may include a mixture of particles of a heat conducting material and of another material.

The method may include controlling the temperature control and fluid supply unit based on a desired fluid temperature and a temperature measured by the temperature sensor.

The method may include sensing the temperature of the fluid by a temperature sensor that may be arranged to contact the fluid that flows within the system.

The method may include sensing a temperature of a fluid without contacting the fluid.

The method may include sensing a temperature of an organ of a patient once a temperature sensor is inserted in the nose upper canal of the patient.

The method may include controlling power supplied to the thermoelectric heat pump.

The method may include dissipating heat from the thermoelectric pump by a fan and a heat sink.

The overall weight of the system may ranges between 1 and 5 kg.

An combination of any of the mentioned above stage can be provided.

### BRIEF DESCRIPTION OF THE DRAWINGS

Further details, aspects, and embodiments of the invention will be described, by way of example only, with reference to the drawings.

FIG. 1 schematically shows a patient, a temperature control and fluid supply unit, a stomach catheter, and a nose catheter according to an embodiment of the invention;

FIG. 2 is a cross sectional view of a patient, a stomach catheter, a nose catheter and areas proximate to each catheter that are directly affected by the temperature of the fluids that flows through each catheter according to an embodiment of the invention;

FIG. 3 is a cross sectional view of a skull of the patient and a nose catheter according to an embodiment of the invention;

FIG. 4 illustrates a nose catheter according to an embodiment of the invention;

FIG. 5 is a cross sectional view of a stomach of the patient and a stomach catheter according to an embodiment of the invention;

FIG. 6 is a stomach catheter according to an embodiment of the invention;

FIG. 7 illustrates a carotid scarf that is placed around the neck of a patient, according to an embodiment of the invention;

FIGS. 8-11 illustrate various portions of the temperature control and fluid supply unit according to various embodiments of the invention;

FIG. 12 illustrates a method for affecting a temperature of a client according to an embodiment of the invention;

FIG. 13 illustrates a method for affecting a temperature of a client according to an embodiment of the invention;

FIG. 14 illustrates a method for affecting a temperature of a client according to an embodiment of the invention;

FIGS. 15-16 and 21 illustrate catheters according to embodiments of the invention;

FIG. 17 illustrates a system and a patient according to an embodiment of the invention;

FIGS. 18 and 19 illustrate a water block according to an embodiment of the invention; and

FIG. 20 illustrates a method for affecting a temperature of a client according to an embodiment of the invention.

### DETAILED DESCRIPTION OF THE DRAWINGS

In the following specification, the invention will be described with reference to specific examples of embodiments of the invention. It will, however, be evident that various modifications and changes may be made therein without departing from the broader spirit and scope of the invention as set forth in the appended claims.

Because the apparatus implementing the present invention is, for the most part, composed of electronic components and circuits known to those skilled in the art, circuit details will not be explained in any greater extent than that considered necessary as illustrated above, for the understanding and appreciation of the underlying concepts of the present invention and in order not to obfuscate or distract from the teachings of the present invention.

Any combination of any of method claims can be provided. For example, any combination of originally filed claim 22 and one or more originally filed claim out of originally filed claims 23-45 can be provided.

Any combination of any system claim can be provided. For example, any combination of originally filed claim 1 and one or more originally filed claim out of originally filed claims 2-21 can be provided.

In the following specification, the invention will be described with reference to specific examples of embodiments of the invention. It will, however, be evident that various modifications and changes may be made therein without departing from the broader spirit and scope of the invention as set forth in the appended claims.

This application incorporates by reference PCT patent application publication WO2009/004635 International filing date Jul. 6, 2008 and in its entirety.

This system is an advanced non-invasive temperature management solution, in the field of medical treatments. This is a breakthrough in medical thermoregulation by helping to control body temperature free of time pressure. This is an unprecedented approach to thermoregulation (controlled temperature management system which allows continuous closed loop feedback and adjustment, based on the individual's physiological responses over a prolonged period).

This system has the dual capability of controlling precisely body temperature in two channels: cooling and warming at the same time. This solution offers the optimal non-invasive system for brain cooling therapy. It is an innovative treatment for cardiac arrest, acute stroke, traumatic brain injury, acute myocardial infraction and sepsis. Cooling therapy addresses the need for Neuro protection by cooling the brain. This system makes available Hypothermia and hyperthermia treatment safe.

This system combines a temperature sensor with controlled unit, which supplies water in the required temperature into the Catheters and Scarf. During operation, water circulates through Catheters and/or Scarf by the thermoregulation unit.

In the following specification, the invention will be described with reference to specific examples of embodiments of the invention. It will, however, be evident that various modifications and changes may be made therein without departing from the broader spirit and scope of the invention as set forth in the appended claims.

Because the apparatus implementing the present invention is, for the most part, composed of electronic components and circuits known to those skilled in the art, circuit details will not be explained in any greater extent than that considered necessary as illustrated above, for the understanding and appreciation of the underlying concepts of the present invention and in order not to obfuscate or distract from the teachings of the present invention.

It has been shown that cooling the brain using a nasal catheter is highly beneficial.

It has been shown that using two independently controllable temperature affecting elements can be used to cool the brain while heating other organs. Conveniently, multiple temperature affecting elements can first cool the patient and after a predefined time (of after reaching a certain temperature) the brain can be maintained at a low temperature by a temperature affecting element (such as a carotid scarf, and additionally or alternatively, a nasal catheter) while another temperature effecting element (such as a stomach catheter) warms other organs of the patient.

Conveniently, the system provides an advanced non-invasive temperature management solution, in the field of medical treatments. This system helps to control body temperature free of time pressure. The system provides controlled temperature management which allows continuous closed loop feedback and adjustment, based on the individual's physiological responses over a prolonged period.

Conveniently, the system can be applied for brain cooling therapy, as well as for treating cardiac arrest, acute stroke, traumatic brain injury, acute myocardial infraction and sepsis.

The system can include one or more temperature sensors, each can be included within or otherwise inserted in proximity to each temperature affecting element.

The system uses a fluid based temperature control mechanism in which fluids can be heater of cooled to desired temperature and then be sent through each temperature affecting element. Conveniently, a temperature sensor can include a naso-gastric tube is inserted through the nose, past the throat and reaches the stomach.

Any of the mentioned above temperature sensors can sense the temperature at different locations of the patient. If, for example, the sensed temperature falls the device can provide heat to the stomach and prevent the possible deleterious effects of total body hypothermia.

The system is small, portable and easily applied and therefore can by used by paramedics immediately even before arrival to the hospital. Applying brain cooling as soon as possible may future improve the neurological outcome. Since the system is portable, therapy can be applied during transfer both before arrival to the hospital but also during transport in the hospital itself between the different departments i.e. ER, radiology, ICU or the operating room. The weight of the system can range between 1-5 kg. This weight may or may not include the weight of a battery or of a controllable power supply.

The system can cool the scalp via a helmet, to the neck using a cooling collar and to the nasal area. By cooling different areas of the head and neck the brain temperature can be lowered quickly and evenly.

### Nose Catheter

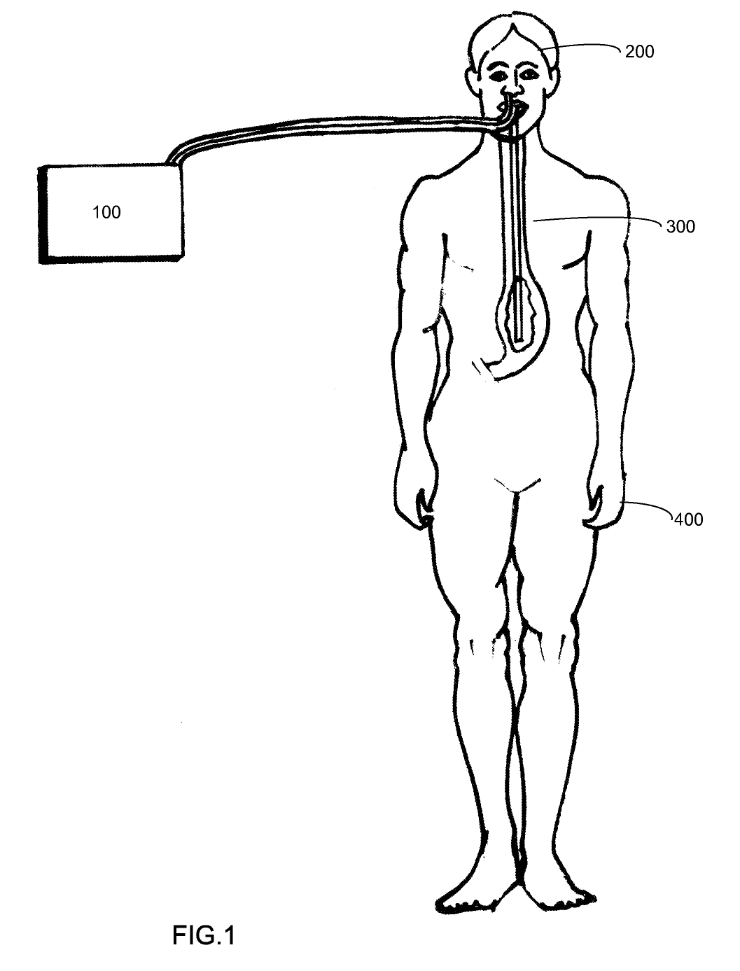

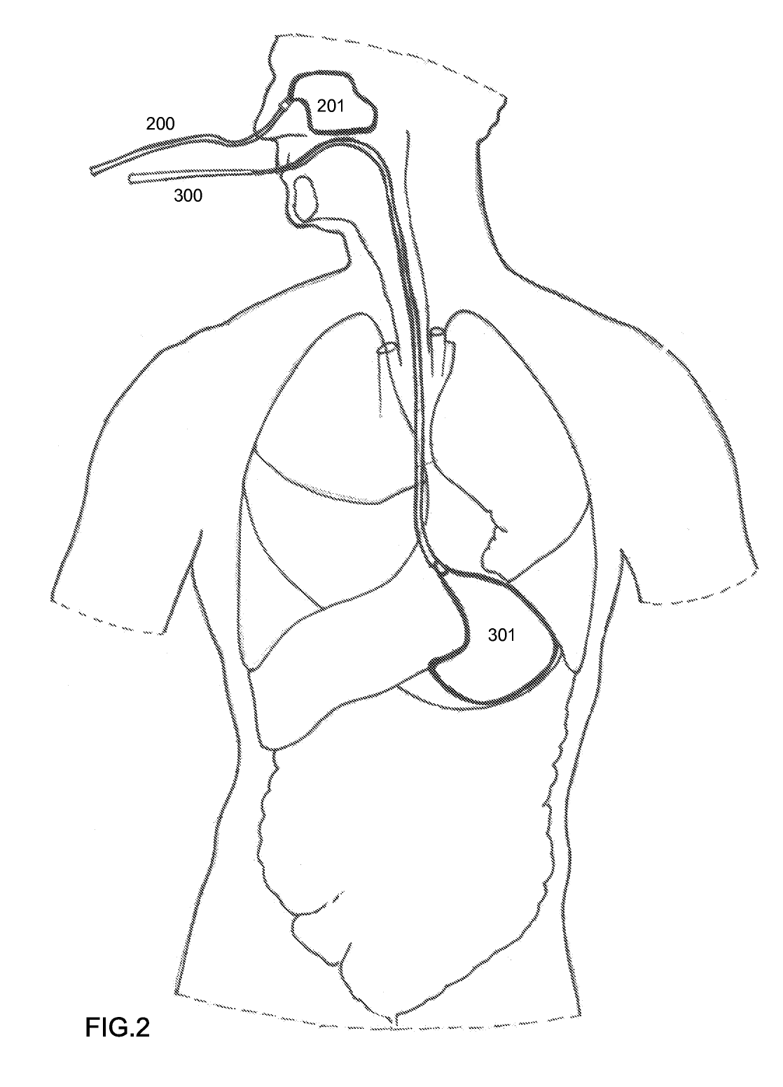

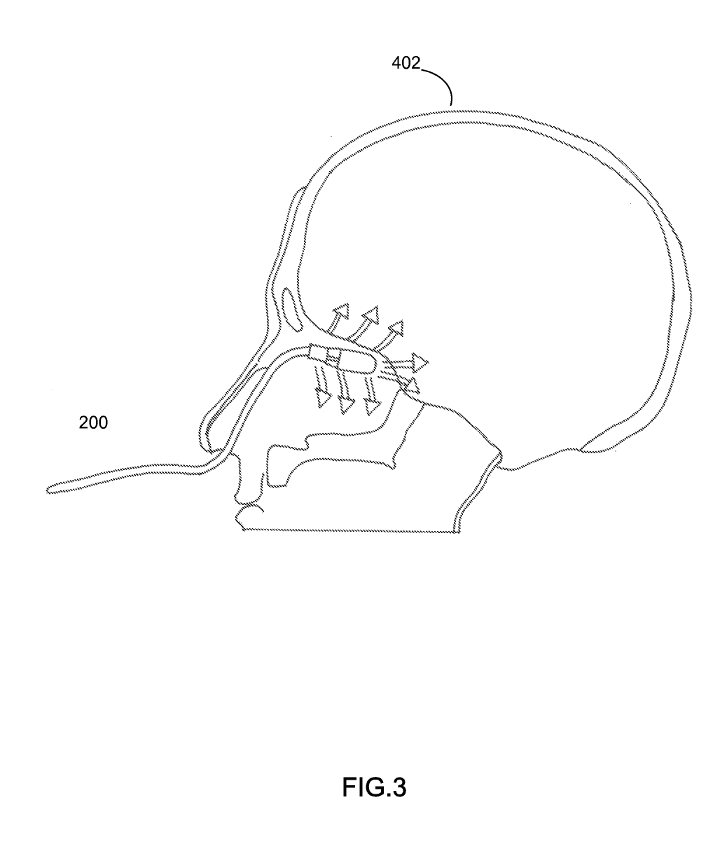

The nose catheter can be a non-invasive single use (disposable) catheter that is inserted into the nose upper channel of a patient. FIG. 1 illustrates nose catheter 200 that is connected to temperature control and fluid supply unit 100. FIG. 2 illustrates the nose catheter 200 as being inserted towards the nose upper channel while FIG. 3 illustrates the nose catheter 200 as being fully inserted through the nose upper channel. It can effectively affect the temperature of the brain-especially cool it.

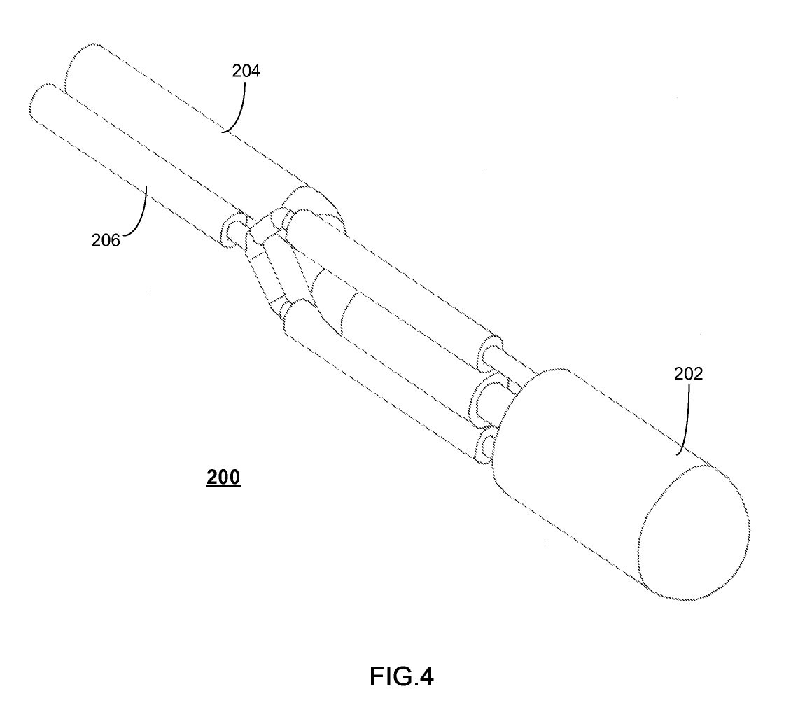

FIG. 4 illustrates nose catheter 200 as including tip such as stainless steel tip 202 that is connected to input and output tubes 204 and 206 that enable the closed loop circulation of fluid (referred to as first fluid) from temperature control and fluid supply unit 100, through tip 202 and towards temperature control and fluid supply unit 100. These tubes can be flexible, made of temperature conductive material.

Stainless steel tip 202 can be covered with silicon (or other interfacing material) which prevents direct contact with inside tissues of the patient. Tip 202 transports heat energy (for example low temperature heat energy) to local blood vessels into face parts and base of skull 402.

Nose catheter 200 is shaped in response to the nose anatomy and prevents direct impact to sensitive tissues. It enables a fast and efficient flow of liquid to keep nasal cavity at desired temperature while preventing constant pressure on Nasal walls.

Either one of input and output tubes 204 and 206 may be an extrude medical grade multi-lumen tube.

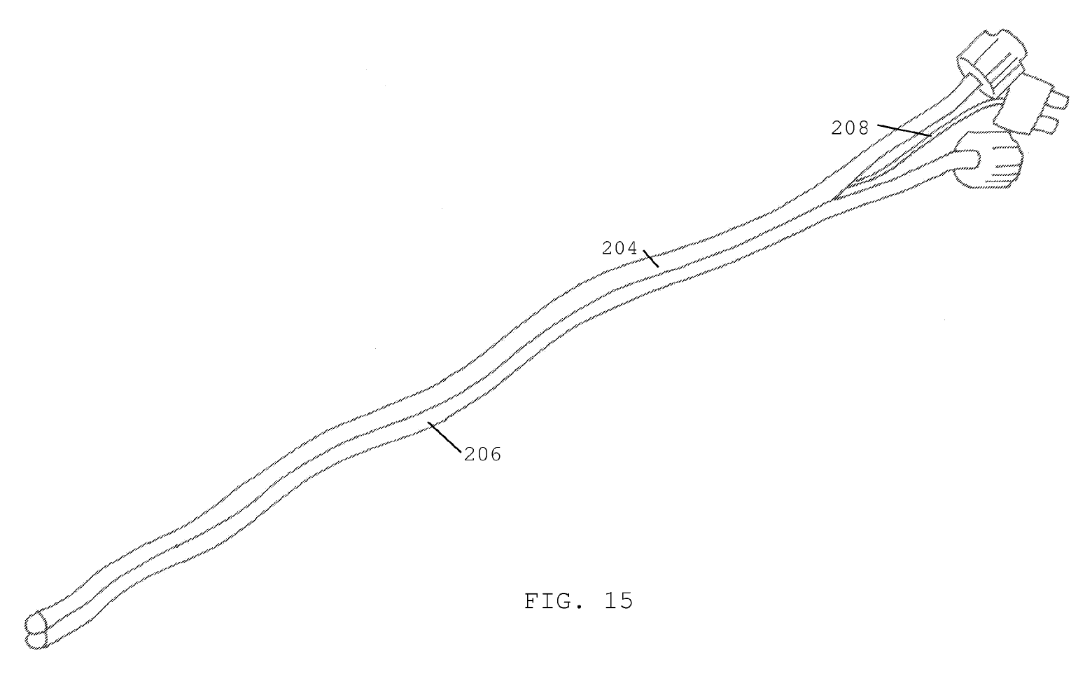

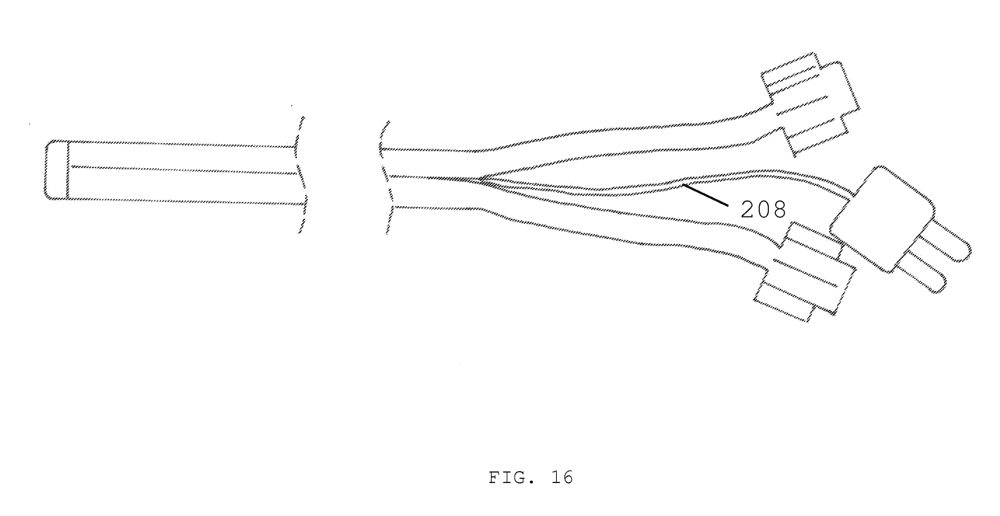

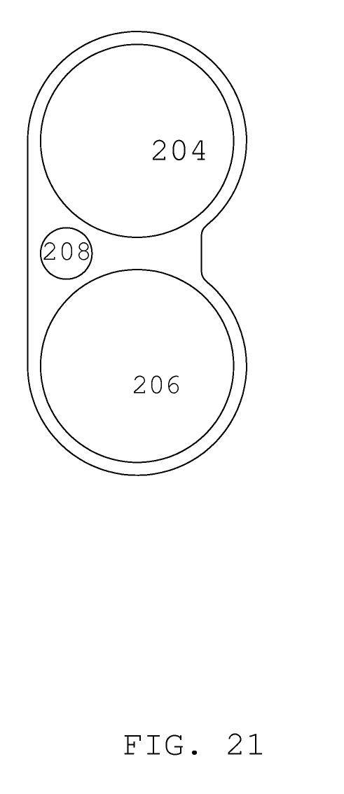

According to an embodiment of the invention the nose catheter 200 may also include a third tube (denoted 208 in FIGS. 15, 16 and 21)—for sensing the temperature of the nasal cavity. FIG. 4 does not illustrate a third tube but such a tube is illustrated in FIGS. 15, 16 and 21. These three tubes are connected to a tip such as the stainless tip illustrated in FIG. 4. The fluid from an input tip contacts the tip (that may be hollow), cools the tip, and flows to the output tube. It is noted that a tube can be used for transferring fluid as well as hosting a temperature sensor. It is noted that there may be multiple input tubes, multiple output tubes and a combination thereof. A large diameter tube can be replaced by multiple narrower tubes. The tip and the tubes can be welded to each other or otherwise connected to allow the flow of fluid from one or more input tube to one or more output tube while affecting the temperature of the tip. The input and output tubes can be a single tubes. The tip can be replaced by other intermediate elements. There can be more than a sing tip. The tip can be split to multiple elements.

### Stomach Catheter

The nose catheter can be a non-invasive single use (disposable) catheter that is inserted through the mouth into the stomach of the patient.

The mid Esophagus lay in the center of chest, near the big vessels and the heart. The distal part of esophagus and the stomach are located in the abdominal cavity and close to the heart (only separated by a diaphragm) and to high blood flow organs such as the liver.

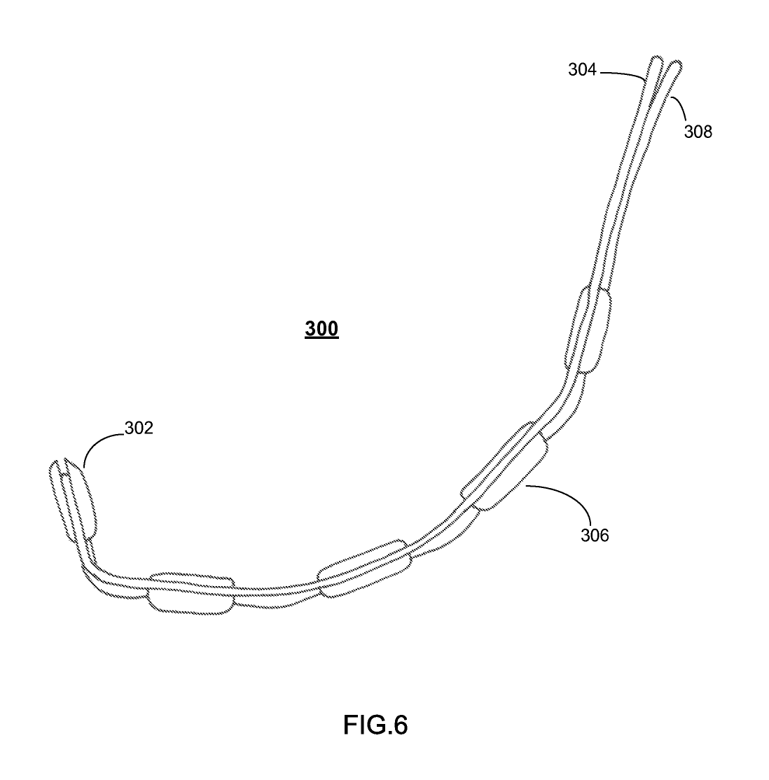

FIG. 1 illustrates a patient 400 and a stomach catheter 300 that is connected to temperature control and fluid supply unit 100. FIGS. 2 and 6 illustrates stomach catheter 300 as being inserted through the mouth towards the stomach of the patient.

Stomach catheter 300 can be inserted into the stomach. It receives fluid (referred to as a second fluid) from temperature control and fluid supply unit 100 via flexible tubes 304 and 308 (of FIG. 6). These tubes can be made of silicone.

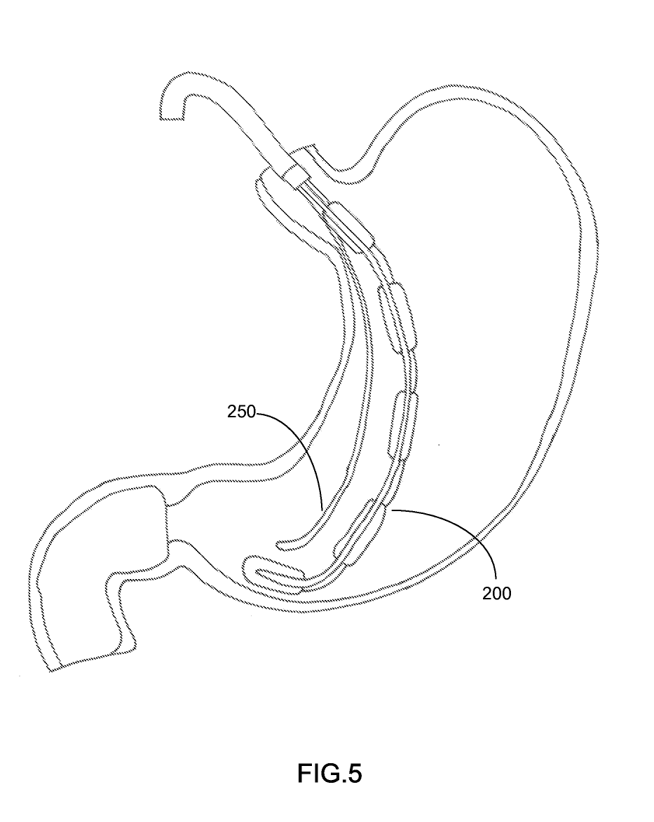

Stomach catheter 300 can transport cooling or heating energy (via passage of the second fluid) to various organs such as the Heart and the Liver. In order to achieve a flexible form the stomach catheter is designed as joints (306 of FIG. 6) connected to two flexible silicon tubes (304 and 308) operating in a close loop unit. A regular Gastric tube (250 in FIG. 5) can be combined with stomach catheter 300 and also connected to temperature control and fluid supply unit 100. The gastric tube can include a temperature sensor.

Either one of the two flexible tubes (304 and 308) can be an extrude medical grade multi-lumen tube and may be made of a mixture of a metal powder and a polymer (or other material) whereas the metal powder may increase the heat conduction.

That tube may be surrounded by another tube—that does not include the metal powder and may prevent the metal powder from contacting internal organs of a patient.

### Carotid Scarf

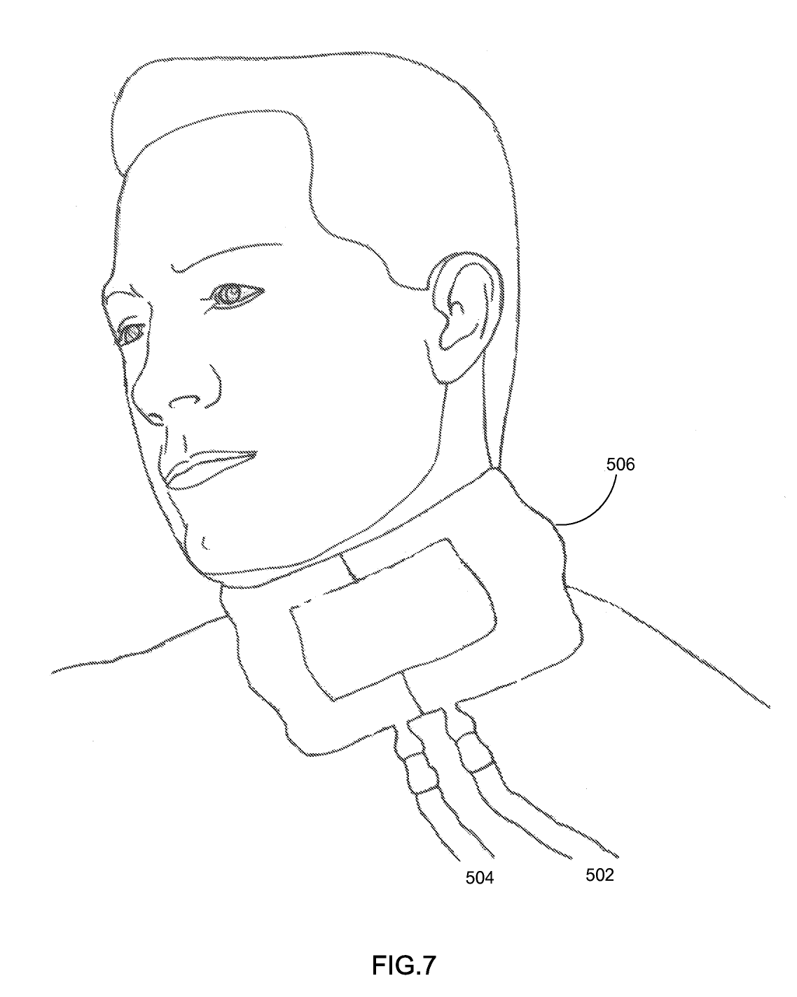

Carotid scarf (denoted 500 in FIG. 7) can be worn over the neck of the patient. It can be used instead of the nose catheter or in combination thereof. During operation, fluid (referred to as third fluid) from temperature control and fluid supply unit 100 flows through the carotid scarf and can affect the temperature of the patient. Especially, the carotid scarf can cool the brain by cooling the main arteries that pass through the neck of the patient.

Carotid scarf 500 receives two tubes 502 and 504—one for providing fluid and the other for draining the fluid.

Carotid scarf can be made of flexible synthetic films and fabrics that form water passages (506 of FIG. 7) in order to keep in continual contact with the patient's skin surface without obstructing access to clinical procedures. The carotid scarf can have a soft layer in contact with patient's skin for comfort. The carotid scarf can be biocompatible and disposable, and can be available in various sizes, ranging from infants to adults.

The temperature of fluid that flows through carotid scarf 500 can be determined in view of a temperature of an organ of the patient.

### Temperature Control and Fluid Supply Unit

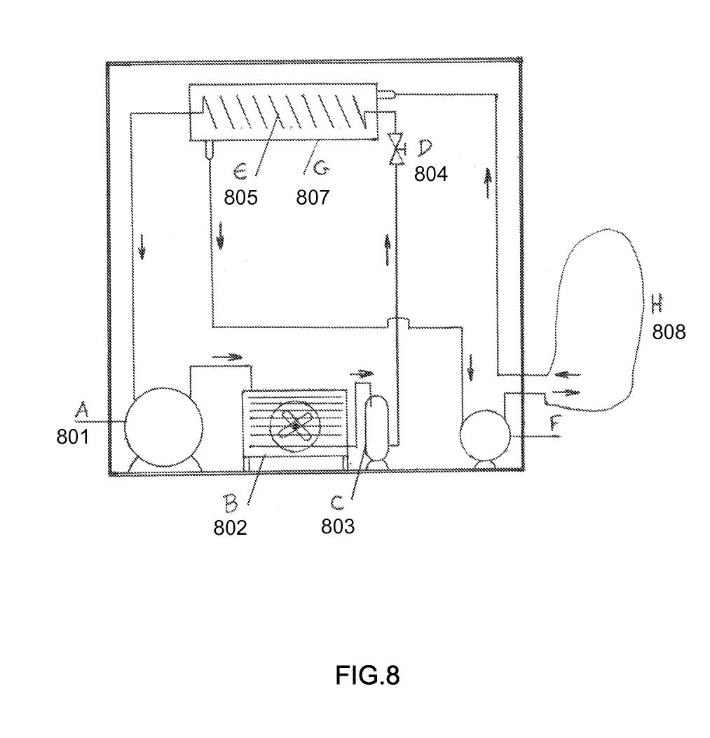

FIGS. 8-11 illustrate various portions of temperature control and fluid supply unit 100 according to various embodiments of the invention.

Temperature control and fluid supply unit 100 is illustrated in FIG. 1 as being connected to nose catheter 200 and to stomach catheter 300.

Temperature control and fluid supply unit 100, when connected to any element out of nose catheter 200, stomach catheter 300 and carotid scarf 500 forms a closed loop in which fluid can flow. While each of these elements (200, 300 and 500) and especially nose catheter 200, stomach catheter 300 are disposable, temperature control and fluid supply unit 100 can be re-sued as it does not contact and even enter the patient.

Temperature control and fluid supply unit 100 can independently control a temperature of fluid that flows through each element out of nose catheter 200, stomach catheter 300 and carotid scarf 500. These fluids are referred to as first, second and third fluids to illustrates the independent control of the temperature of each fluid. It is noted that this is not necessarily so and that according to another embodiment of the invention two or more of the mentioned above elements (200, 300, 500) can receive the same fluid that has the same temperature.

Temperature control and fluid supply unit 100 can determine the temperature of each fluid according to one or more parameters including but not limited to sensed temperature of an organ of a patient, medical history of the patient, temperature setting information indicative of desired fluid temperature per one or more parameters including patient age, patient weight, patient sex, patient status, and the like. The temperature setting information can include desired temperature changes over time.

Various prior art methods and systems can be applied for cooling or heating a fluid and for provided the cooled or heated fluid.

Conveniently, temperature control and fluid supply unit 100 is compact and can be easily carried or included in ambulances.

FIG. 8 illustrates a fluid refrigerating unit of temperature control and fluid supply unit 100 according to an embodiment of the invention.

Referring to FIG. 8, the fluid refrigerating unit includes: (A) Compressor 801, (B) Condenser 802, (C) Receiver 803, (D) Expansion valve 804, (E) Refrigerating coil 805, (F) Fluid pump 806, (G) Evaporator/Water tank 807, and (H) Closed loop water/fluid piping 808.

The compressor draws the refrigerant in the vapor phase from the evaporator or cooler and discharges it to the condenser at an increased temperature and pressure. Refrigerant reaches expansion valve as fluid and discharged into evaporator sprayed. There refrigerant evaporates and absorbs heat during the process from the cold body at the lowest temperature in the cycle.

The refrigerating process functions electrically and automatically by system's pre-adjustment for cold fluid supply at a stable temperature or by a manual instruction in view of changes in demands.

The pre-adjustment of the expansion valve and the thermostat (that can be regulated manually any time) keep the required temperatures from distortion.

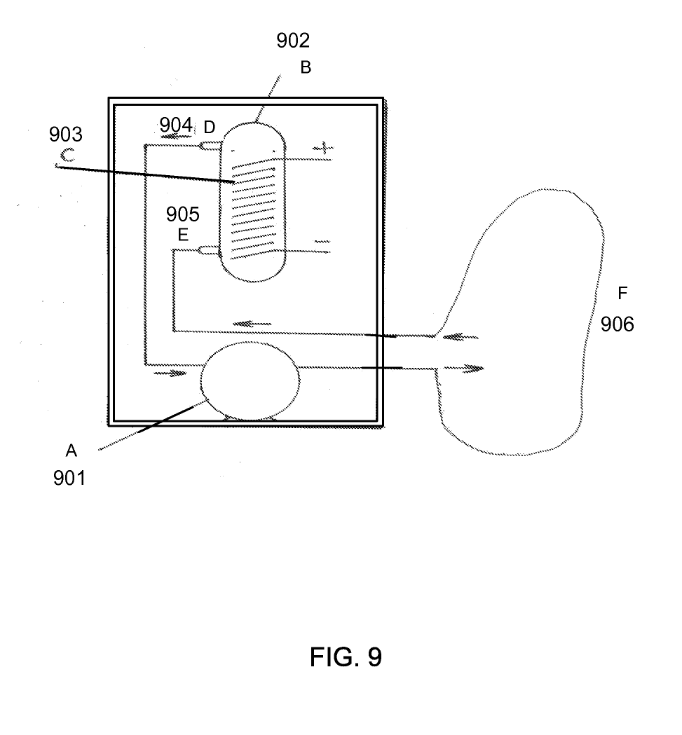

FIG. 9 illustrates a fluid heating unit of temperature control and fluid supply unit 100 according to an embodiment of the invention.

The fluid heating unit is close looped and contains several basic elements: (A) liquid pump 901, (B) a small water tank 902, (C) a high capacity electric element 903, (D) outlet of hot liquid 904, (E) inlet of returning liquid 905, and (F) closed loop water/fluid piping 906.

The small water tank contains the electric element and two openings.

The inlet and outlet of fluid—the fluid pump pumps hot fluid from the tank and discharges it by the closed loop piping to designated instruments and return in a loop into tank.

The heating process functions electrically and automatically by systems pre-adjustment for hot fluid supply at a stable temperature or by manual instruction in view of changes in demands. The pre-adjustment is by thermostat.

Referring to FIG. 9, the fluid heating unit includes: (i) Fluid pump pumps from upper part of fluid tank and discharges hot fluid by the closed loop piping to designated instruments and return in a loop into lower part of the tank. (ii) A small volume fluid tank (in order to decrease time of heating); (iii) An immersed electric element that heats fluid in tank in the demanded level of temperature and which is guarded by a thermostat; (iv) Outlet of hot fluid; (v) Inlet of returning fluid by loop piping; and (vi) Loop piping.

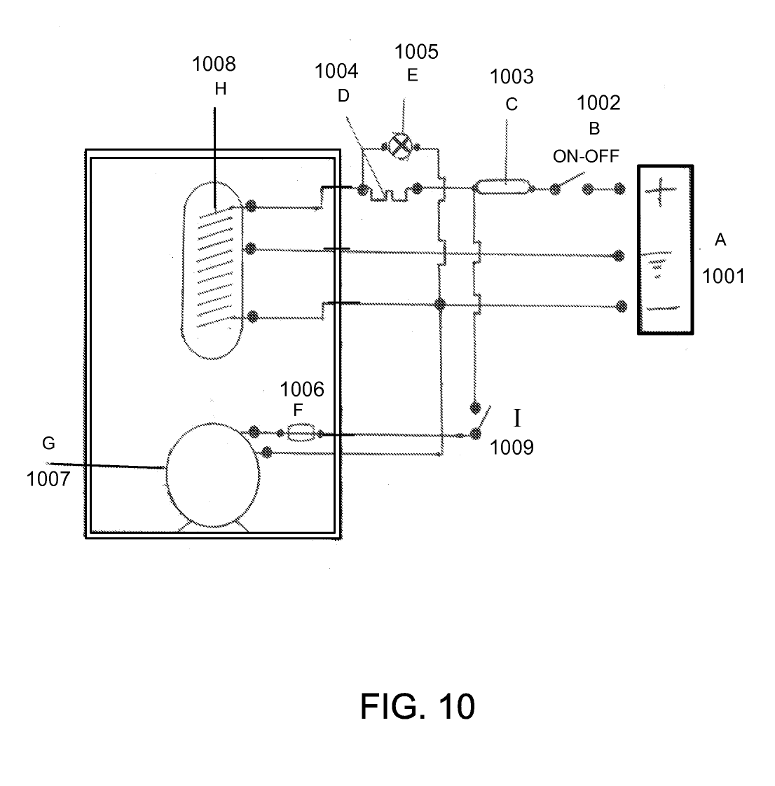

FIG. 10 provides an electrical scheme of the fluid heating unit.

It includes: Direct power supply 12 v (A) 1001, on/off main switch 1002, Fuse 1003, Thermostat 1004, Light emitting diode 1005, Flow switch 1006, Pump 1007, Heater element 1008, and discharging fluid switch to closed loop piping.

By operating the main switch (B) 1002 the system is under current.

The fluid in tank is cold, so thermostat's probe (D) 1004 enables current reach the heater element (H) 1008 and led (E) 1005 lights up. While fluid reaches the right temperature level, thermostat's probe (D) 1004 causes current cut down and heating stops. Likewise led (E) 1005 goes out.

When hot fluid supply is required a switch 1009 must be pressed (I) and by that, flow switch (F) 1006 causes the pump (G) 1007 operate and discharge fluid.

As long as the switch (I) 1009 will be pressed, hot fluid will keep flowing into closed loop piping.

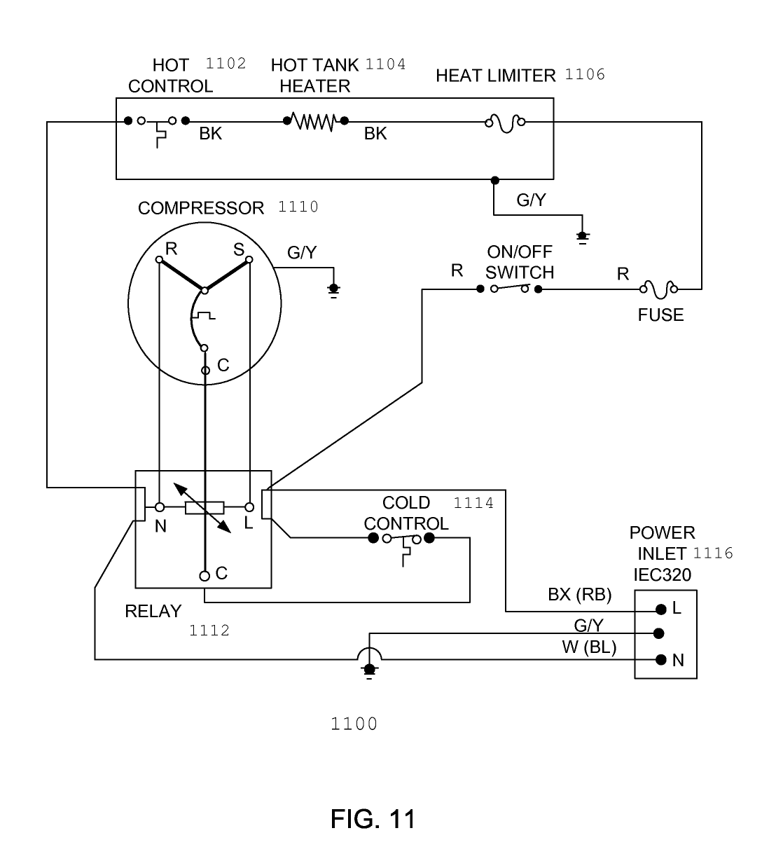

FIG. 11 illustrates an electrical schema of the system. It illustrates some of the elements of the previous figures. For example, it illustrates a three phase compressor, that can be fed by a relay that is controller by a cold circuit switch. It also illustrates a hot control switch that can selectively connect or disconnect hot tank heater (represented by a resistor) to the supply power. A heat limiter and fuse protect the elements of the system from over currents. In addition an ON/OFF switch can turn the system on or off.

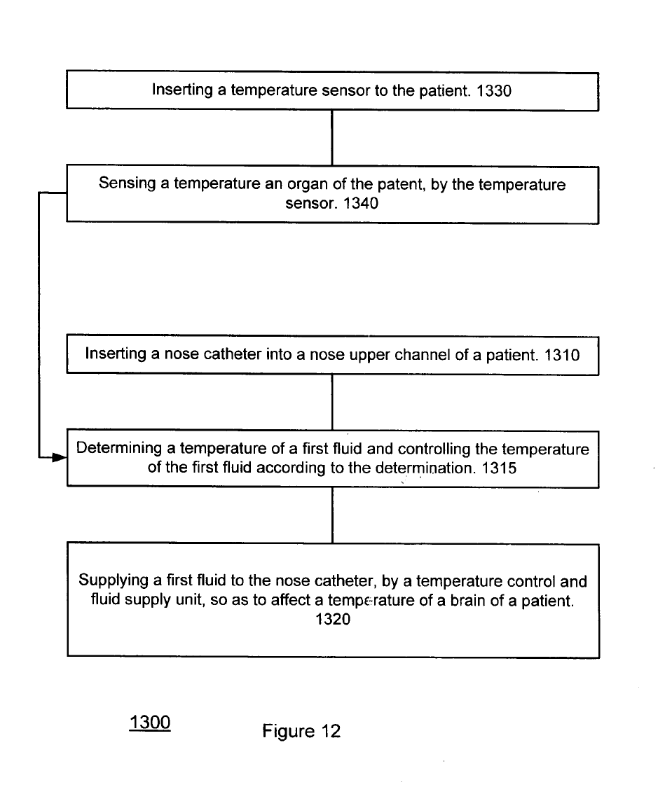

FIG. 12 illustrates method 1300 for affecting a temperature of a patient, according to an embodiment of the invention.

Method 1300 starts by stage 1310 of inserting a nose catheter into a nose upper channel of a patient. Stage 1310 can include inserting a nose catheter that includes a stainless steel tip. Stage 1310 can be preceded by coating the tip with silicon or other barrier.

Stage 1310 is followed by stage 1315 of determining a temperature of a first fluid and controlling the temperature of the first fluid according to the determination.

Stage 1315 is followed by stage 1320 of supplying a first fluid to the nose catheter, by a temperature control and fluid supply unit, so as to affect a temperature of a brain of a patient. Stage 1320 can include supplying the first fluid to the nose catheter through an inlet and draining the fluid through the outlet.

Stages 1315 and 1320 can be executed during a long period of time. The temperature can be dynamically changed over time.

Conveniently, stage 1320 includes supplying the first fluid to the nose catheter so as to cool the brain of the patient.

Method 1300 can also includes stage 1330 of inserting a temperature sensor to the patient. Stage 1330 is followed by stage 1340 of sensing a temperature an organ of the patent, by the temperature sensor. Stage 1340 can be followed by stage 1315 wherein the determining of the temperature of the first fluid is responsive to temperature sensed by the temperature sensor. It is noted that method 1300 can include inserting more than a single temperature sensor and determining the temperature of the first fluid in response to multiple sensed temperatures.

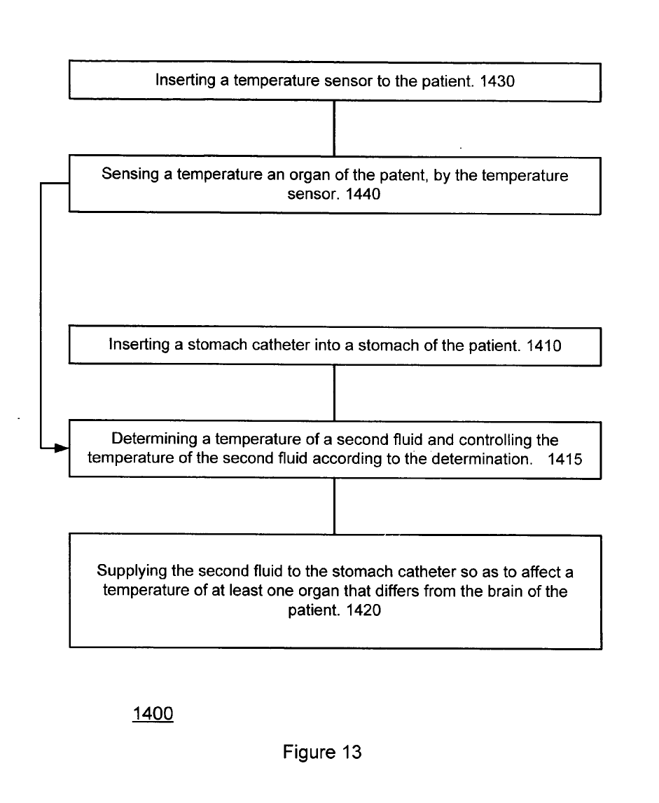

FIG. 13 illustrates method 1400 for affecting a temperature of a patient, according to an embodiment of the invention.

Method 1300 starts by stages 1310 and 1410.

Stage 1310 is followed by stage 1315. Stage 1315 is followed by stage 1320.

Stage 1410 includes inserting a stomach catheter into a stomach of the patient.

Stage 1410 is followed by stage 1415 of determining a temperature of a second fluid and controlling the temperature of the second fluid according to the determination.

Stage 1415 is followed by stage 1420 of supplying the second fluid to the stomach catheter so as to affect a temperature of at least one organ that differs from the brain of the patient. This organ can be the liver, the heart, the stomach and the like.

It is noted that one or more temperature sensors can sense the temperature of these one or more other organs and that stage 1415 can be responsive to the sensed temperature—as illustrated by stage 1430 and 1440.

Stages 1315 and 1415 can facilitate controlling temperatures of the first and second fluids so that during at least one period of time, the temperature of the first fluid differs from the temperature of the second fluid.

Stages 1315 and 1415 can facilitate controlling temperatures of the first and second fluids so that during at least one other period of time, the temperature of the first fluid substantially equals the temperature of the second fluid.

Stages 1315 and 1415 can facilitate controlling temperatures of the first and second fluids so that during a first period of time, the stomach catheter and the nasal catheter cool organs of the patient and during a second period of time that followed the first period of time the nasal catheter cools the brain of the patient while the stomach catheter warms at least one other organ of the patient.

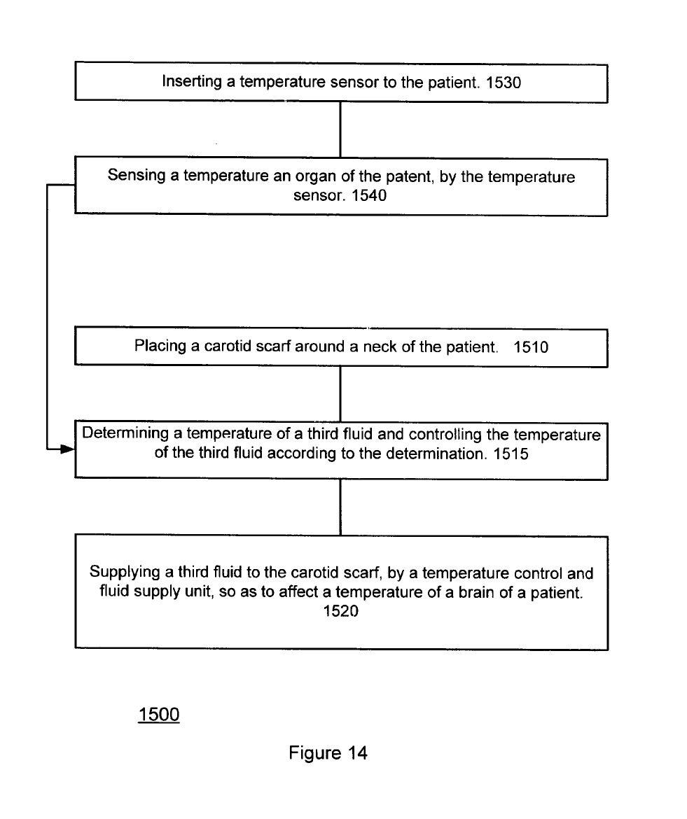

FIG. 14 illustrates method 1500 for affecting a temperature of a patient, according to an embodiment of the invention.

Method 1500 starts by stage 1510 of placing a carotid scarf around a neck of the patient.

Stage 1510 is followed by stage 1515 of determining a temperature of a third fluid and controlling the temperature of the third fluid according to the determination.

Stage 1515 is followed by stage 1520 of supplying a third fluid to the carotid scarf, by a temperature control and fluid supply unit, so as to affect a temperature of a brain of a patient.

Method 1500 can also includes stages 1530 and 1540 that are analogues to stage 1330 and 1340.

Conveniently, method 1500 also includes at least one sequence of stages of method 1300 or 1400.

It is noted that any combination of the above method methods or stages can be applied. For example, a method can include utilizing a combination of nose catheter, carotid scarf and stomach catheter. Yet for another example, only a combination of carotid scarf and stomach catheter can be utilized. Yet for a further example, a head helmet can be used in addition to any method.

According to an embodiment of the invention a portable brain cooling system is provided.

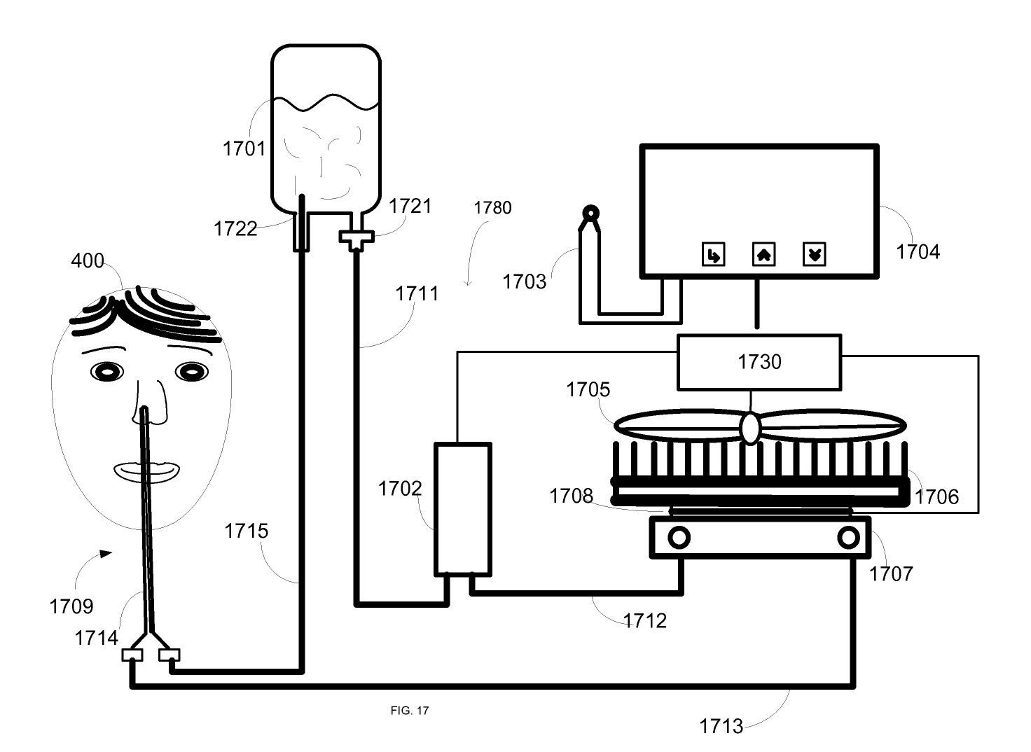

FIG. 17 illustrates system 1700 and a patient 400 according to an embodiment of the invention.

System 1700 includes a temperature control and fluid supply unit 1780 and a nose catheter 1709.

The temperature control and fluid supply unit 1780 includes a thermoelectric heat pump 1708, a heat sink 1706, a fluid block 1707, a fan 1705, a controller 1704, a temperature sensor 1703, a fluid pump 1702, tubes such as tubes 1711-1715 and a power supply unit such as controllable power source 1730.

The temperature control and fluid supply unit 1780 may include a fluid source or may be connected to such a fluid source.

The system 1700 includes tubes 1711, 1712, 1713, 1714 and 1715. These tubes and other elements of the system form a close loop in which fluid circulates. The fluid is cooled by fluid block 1707 and is circulated by fluid pump 1702.

Tube 1711 is connected to (or has) an input element 1721. Tube 1715 has (or is connected to) output element 1725. Input element 1721 is connected between infusion bag 1701 (or an output of another fluid source). Tube 1711 is connected between the input element 1721 and the fluid pump 1702. Tube 1712 is connected between the fluid pump 1702 and an input of the fluid block 1707. Tube 1713 is connected between an output of the fluid block 1707 and an input tube of the nose catheter 1709.

Tube 1714 is connected between an output tube of the nose catheter 1709 and an input of the infusion bag 1701 (or an input of another fluid source).

The fluid pump 1702 circulates the fluid in these tubes and other elements.

It is noted that the number of tubes, the location of elements of system 1700 and the number of elements can differ from those illustrated in FIG. 17. For example, a single tube can replace tubes 1712 and 1713—and such single tube can pass through a space formed in the fluid block 1701. Yet for another example—the fluid pump 1702 can be located after the fluid block 1707.

The system 1700 provides an advanced non-invasive temperature management solution, in the field of medical treatments. The system provides a breakthrough in medical thermoregulation by helping to control body temperature free of time pressure. The system implements an unprecedented approach to thermoregulation (controlled temperature management system which allows continuous closed loop feedback and adjustment, based on the individual's physiological responses over a prolonged period).

This system may be designed for brain cooling & supporting TBI (Trauma Brain Injuries) out of Hospital treatment in the field for handling by the following: Ambulance Paramedic, Flying doctors, Front line Hospital, Military doctors & medical assistant.

According to various embodiments of the invention the system is very portable and can operate for relatively long periods by using small energy sources or any other energy sources available to medical staff.

According to an embodiment of the invention the system circulates fluids from an infusion bag. The system has: (A) a fluid input that can be connected to the output of the catheter bag (or to an output of any interfacing elements that are connected to the output of the infusion bag—such as tubes, valves and the like) and (B) a fluid output that can be inserted into the infusion bag—the fluid output can include a hollow and sharp needle that can be inserted into the infusion bag. Using the infusion bag as a source of liquid simplifies the operation of the system as medical staff is equipped with infusion bags and also provides a sterile source of fluid that does not contaminate the system and eases the maintenance of the system. The infusion bag can include fluids of various types. The amount of fluid to be used may be relatively small and an infusion bag can provide the desired amount of fluids.

According to another embodiment of the invention the system can include (or use) another source of fluid—such as a fluid tank.

The system can use compact and highly efficient cooling and heat exchange elements such as (a) thermoelectric heat pump 1708 (also known as a Peltier device or a Peltier heat pump), (b) a heat sink 1706 (also referred to as a heat exchanger), (c) a fluid block 1707 and (d) a fan 1705.

The thermoelectric heat pump 1708 transfers heat from a first side (cold side) to another side (hot side). The cold side is connected to the fluid block 1707 that in turn cools fluid that flows through the fluid block. The fluid block is made of heat conducting material such as metal and allows the cold side of the thermoelectric heat pump 1708 to affect fluid that passes through the fluid block 1707.

The heat sink 1706 is connected to the hot side of the thermoelectric heat pump 1708 and dissipates heat developed on the hot side. The heat sink 1706 can be cooled by the fan 1705. The heat sink 1706 can be of various shapes and especially shapes that have large surface area. The heat sink 1706 can include multiple fins that are spaced apart from each other and a fin base that may be normal to the fins.

The fan 1705 can be any commercially available fan and especially fans that have a flat cage and be relatively small. For example the fan 1705 can be about 11.9 cm long, 11.9 cm wide and 26 cm deep. The inventors used a fan out of the 4710KL fan family of NMB Technologies Corporation of the United States. Different fans of this family can be fed by 12 Volts or 24 Volts, may consume between 1.44 watts and 6.96 watts, weigh about 180 grams and produce maximal air flows between 1.8 to 3.25 (m³/min)—depending upon the model of the fan.

The fan 1705, thermoelectric heat pump 1708 and fluid pump 1702 can be controller by controller 1704. The controller 1704 can have a man machine interface and may include a display 1711 and control buttons 1712. The man machine interface can include soft control buttons (having a programmable function) or hard control buttons or a combination thereof. The buttons are optional and can be replaced by a display 1711 that is a touch screen. The controller 1704 may be connected to a temperature sensor 1703 that senses the temperature of the fluid that passes through the system or may sense the nasal canal temperature. In order to sense the temperature of the fluid the sensor can be inserted into any of the tubes 1712-1715 or may contact these tubes (or at least a heat conducting element of the tubes).

The controller 1704 allows a user to insert cooling parameters such as a desired temperature and then may control the fan 1705, the thermoelectric heat pump 1708 and the fluid pump 1702 to achieve the desired temperature. According to an embodiment of the invention the fan 1705, the thermoelectric heat pump 1708 receive power from a controllable power source 1730 and the controller controls the voltage (or current) provided to these elements and thus controls their operation. Typically, higher supply voltages result in better performance—faster fan speed, increased temperature difference between the cold side and the hot side of the thermoelectric heat pump 1708, faster flow of fluid.

The nose catheter 1709 can be equivalent to nose catheter 200 of any of the previous figures.

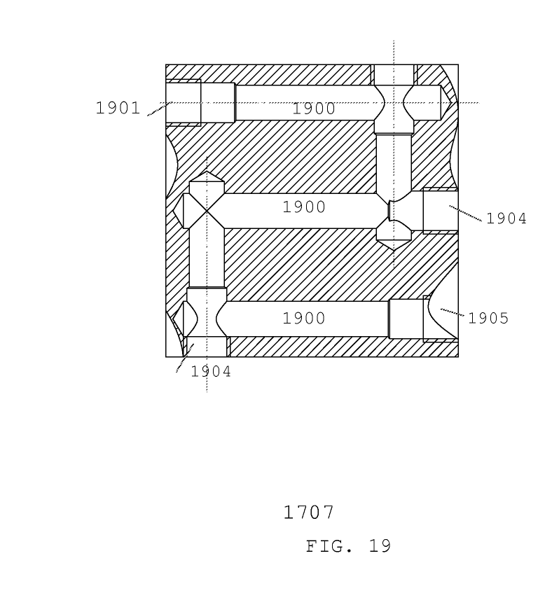

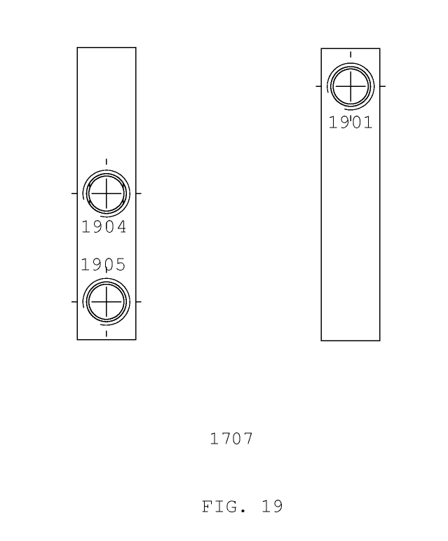

FIGS. 18 and 19 illustrate a fluid block 1707 according to an embodiment of the invention. Multiple drills 1900 made within a metal block 1902 form a serpentine pattern in which fluid passes while being cooled. The serpentine pattern starts at input 1901 and ends at output 1905. Input 1901 is made by one drill while output 1905 is made by another. Additional drills that provide portions of the serpentine pattern are sealed by plugs 1904. FIG. 18 is a cross sectional view while FIG. 19 is a side view. It is noted that the serpentine pattern can be replaced by other (linear or non-linear) patterns.

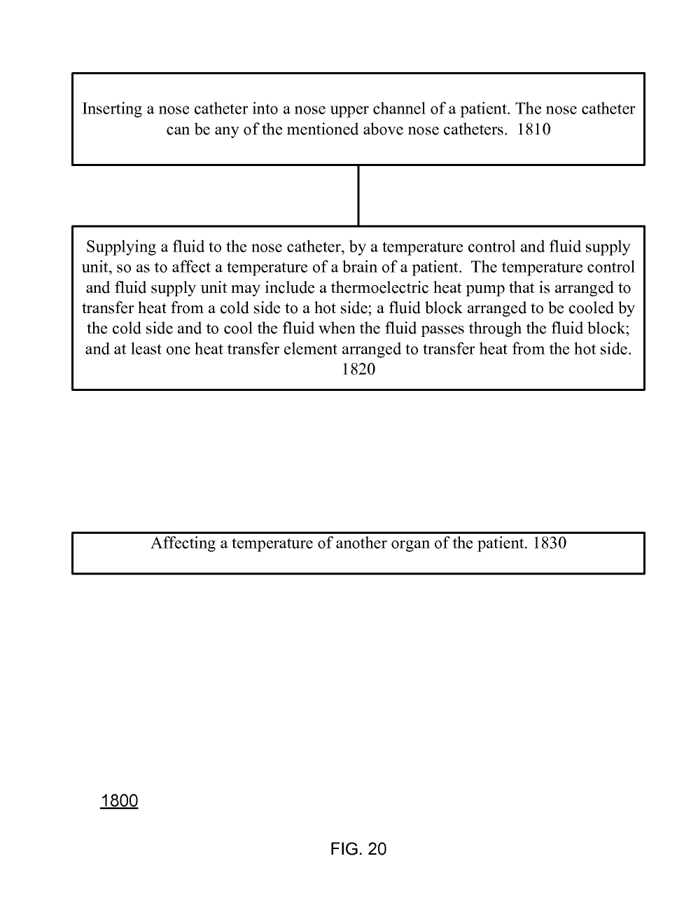

FIG. 20 illustrates method 1800 according to an embodiment of the invention.

Method 1800 includes stage 1810 of inserting a nose catheter into a nose upper channel of a patient. The nose catheter can be any of the mentioned above nose catheters.

Stage 1810 is followed by stage 1820 of supplying a fluid to the nose catheter, by a temperature control and fluid supply unit, so as to affect a temperature of a brain of a patient. The temperature control and fluid supply unit may include a thermoelectric heat pump that is arranged to transfer heat from a cold side to a hot side; a fluid block arranged to be cooled by the cold side and to cool the fluid when the fluid passes through the fluid block; and at least one heat transfer element arranged to transfer heat from the hot side.

Stage 1820 may include at least one of the following:
1. Activating the thermoelectric heat pump, dissipating heat from the hot side of the thermoelectric heat pump and allowing the cold side to cool the fluid.
2. Supplying the fluid to the nose catheter so as to cool the brain of the patient.
3. Receiving the fluid from an infusion bag and retuning the fluid to the infusion bag via an output element. The fluid passes through the nasal catheter before being outputted to the infusion bag.
4. Returning the fluid to the infusion bag via an output element that is a hollow and sharp needle that is arranged to puncture the infusion bag.
5. Allowing the fluid to flow through at least one tube of the nasal catheter wherein at least one of the input tube and the output tube comprises a mixture of a metal powder and at least one other material.
6. Controlling the temperature control and fluid supply unit based on a desired fluid temperature and a temperature measured by the temperature sensor.
7. Measuring the temperature of the fluid by a temperature sensor that is arranged to contact fluid within the system and sense the temperature of the fluid.
8. Measuring the temperature of the fluid by a temperature sensor that is arranged to sense a temperature of fluid without contacting the fluid.
9. Measuring by the temperature sensor a temperature of an organ of a patient once inserted in the nose upper canal of the patient.
10. Controlling power supplied to the thermoelectric heat pump.
11. Dissipating heat from the thermoelectric pump by a fan and a heat sink.

Stage 1810 may include inserting a temperature sensor to the patient and stage 1820 may include sensing a temperature an organ of the patent, by the temperature sensor; and determining the temperature of the fluid in response to temperature sensed by the temperature sensor.

Method 1800 may include stage 1830 of affecting a temperature of another organ of the patient.

Stage 1830 may include at least one of the following:
1. Inserting a stomach catheter into a stomach of the patient.
2. Supplying a second fluid to the stomach catheter so as to affect a temperature of at least one organ that differs from the brain of the patient.
3. Controlling temperatures of the fluid and of the second fluids so that during at least one period of time, the temperature of the first fluid differs from the temperature of the second fluid.
4. Controlling temperatures of the fluid and of the second fluid so that during at least one other period of time, the temperature of the fluid substantially equals the temperature of the second fluid.
5. Controlling temperatures of the fluid and of the second fluid so that during a first period of time, the stomach catheter and the nasal catheter cool organs of the patient and during a second period of time that followed the first period of time the nasal catheter cools the brain of the patient while the stomach catheter warms at least one other organ of the patient.
6. Placing a carotid scarf around a neck of the patient; and supplying a third fluid to the carotid scarf, by a temperature control and fluid supply unit, so as to affect a temperature of a brain of a patient.

However, other modifications, variations, and alternatives are also possible. The specifications and drawings are, accordingly, to be regarded in an illustrative rather than in a restrictive sense.

The word ‘comprising’ does not exclude the presence of other elements or steps then those listed in a claim.

Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

We claim:

1. A system for affecting a temperature of a patient, the system comprises:
a nose catheter that is adapted to be inserted into a nose upper channel and to receive a fluid; and
a temperature control and fluid supply unit that is adapted to supply the fluid and control a temperature of the fluid so as to affect a temperature of a brain of a patient when the nose catheter is inserted into the nose upper channel of a patient and receives the first fluid;
wherein the temperature control and fluid supply unit comprises:
a thermoelectric heat pump that is arranged to transfer heat from a cold side to a hot side;
a fluid block arranged to be cooled by the cold side and to cool the fluid when the fluid passes through the fluid block; and
at least one heat transfer element arranged to transfer heat from the hot side.

2. The system according to claim 1, comprising an input element for receiving fluid from an infusion bag and an output element for inserting the fluid into the infusion bag.

3. The system according to claim 2, wherein the output element is a hollow and sharp needle that is arranged to puncture the infusion bag.

4. The system according to claim 1, wherein the nose catheter comprises an input tube, an output tube and a tip that are in fluid communication with each other; wherein the input tube is arranged to receive the fluid and to allow the fluid to cool the tip and to exit through the output tube.

5. The system according to claim 1, wherein at least one of the input tube and the output tube comprises a mixture of a metal powder and at least one other material.

6. The system according to claim 5, wherein the metal powder is placed such as not to contact an organ of the patient once inserted in the nose upper canal of the patient.

7. The system according to claim 5, wherein the at least one tube comprises an inner lumen that comprises the metal powder and an outer lumen that surrounds the inner lumen.

8. The system according to claim 1, wherein at least one of the input tube and the output tube comprises a mixture of particles of a heat conducting material and of another material.

9. The system according to claim 1, comprising a controller and a temperature sensor, wherein the controller is arranged to control the system based on a desired fluid temperature and a temperature measured by the temperature sensor.

10. The system according to claim 9, wherein the temperature sensor is arranged to contact fluid within the system and sense the temperature of the fluid.

11. The system according to claim 9, wherein the temperature sensor is arranged to sense a temperature of fluid without contacting the fluid.

12. The system according to claim 9, wherein the temperature sensor is arranged to sense a temperature of an organ of a patient once inserted in the nose upper canal of the patient.

13. The system according to claim 1 comprising a controllable power source arranged to control power supplied to the thermoelectric heat pump.

14. The system according to claim 1, wherein the at least one heat transfer element comprise a fan and a heat sink.

15. The system according to claim 1, wherein an overall weight of the system ranges between 1 and 5 Kilograms.

16. The system according to claim 1, wherein the temperature control and fluid supply unit is adapted to supply the fluid and control the temperature of the fluid so as to cool the brain of a patient when the nose catheter is inserted into the nose upper channel of a patient and receives the first fluid.

17. The system according to claim 1, further comprising a stomach catheter that is adapted to be inserted into a stomach of the patient and to receive a second fluid; and wherein the temperature control and fluid supply unit is further adapted to supply the second fluid and control a temperature of the second fluid so as to affect a temperature of at least one organ that differs from the brain of the patient when the stomach catheter is inserted into the nose upper channel of the patient and receives the second fluid.

18. The system according to claim 17 wherein the temperature control and fluid supply unit is adapted to control temperatures of the fluid and of the second fluid so that during at least one period of time, the temperature of the fluid differs from the temperature of the second fluid.

19. The system according to claim 18 wherein the temperature control and fluid supply unit is adapted to control temperatures of the first and second fluids so that during at least one other period of time, the temperature of the first fluid substantially equals the temperature of the second fluid.

20. The system according to claim 19, wherein the temperature control and fluid supply unit is adapted to control temperatures of the first and second fluids so that during a first period of time, the stomach catheter and the nasal catheter cool organs of the patient and during a second period of time that followed the first period of time the nasal catheter cools the brain of the patient while the stomach catheter warms at least one other organ of the patient.

21. The system according to claim 1 further comprising a carotid scarf that is adapted to be surround a neck of the patient and to receive a third fluid; and wherein the temperature control and fluid supply unit is further adapted to supply the third fluid and control a temperature of the second fluid so as to affect a temperature the brain of the patient when the stomach catheter is inserted into the nose upper channel of the patient and receives the second fluid.

22. A method for affecting a temperature of a patient, the method comprises:
inserting a nose catheter into a nose upper channel of a patient;
supplying a fluid to the nose catheter, by a temperature control and fluid supply unit, so as to affect a temperature of a brain of a patient;
wherein the temperature control and fluid supply unit comprises:
a thermoelectric heat pump that is arranged to transfer heat from a cold side to a hot side;
a fluid block arranged to be cooled by the cold side and to cool the fluid when the fluid passes through the fluid block; and
at least one heat transfer element arranged to transfer heat from the hot side.

23. The method according to claim 22, comprising supplying the fluid to the nose catheter so as to cool the brain of the patient.

24. The method according to claim 22 further comprising:
inserting a temperature sensor to the patient;
sensing a temperature an organ of the patent, by the temperature sensor; and
determining the temperature of the fluid in response to temperature sensed by the temperature sensor.

25. The method according to claim 22, further comprising inserting a stomach catheter into a stomach of the patient; and supplying a second fluid to the stomach catheter so as to affect a temperature of at least one organ that differs from the brain of the patient.

26. The method according to claim 25, further comprising controlling temperatures of the first and second fluids so that during at least one period of time, the temperature of the first fluid differs from the temperature of the second fluid.

27. The method according to claim 26, further comprising controlling temperatures of the fluid and of the second fluid so that during at least one other period of time, the temperature of the fluid substantially equals the temperature of the second fluid.

28. The method according to claim 27, further comprising controlling temperatures of the fluid and of the second fluid so that during a first period of time, the stomach catheter and the nasal catheter cool organs of the patient and during a second period of time that followed the first period of time the nasal catheter cools the brain of the patient while the stomach catheter warms at least one other organ of the patient.

29. The method according to claim 22, further comprising placing a carotid scarf around a neck of the patient; and supplying a third fluid to the carotid scarf, by a temperature control and fluid supply unit, so as to affect a temperature of a brain of a patient.

30. The method according to claim 29, further comprising inserting a stomach catheter into a stomach of the patient; and supplying a second fluid to the stomach catheter so as to affect a temperature of at least one organ that differs from the brain of the patient.

31. The method according to claim 22, further comprising inserting a stainless steel tip of the nasal catheter into the nose upper channel, providing the stainless steel tip first fluid via an inlet and draining the first fluid from the stainless steel tip through an outlet.

32. The method according to claim 22 comprising cooling the fluid by the cold side and transferring heat from the hot side by the at least one heat transfer elements.

33. The method according to claim 22, comprising receiving the fluid from an infusion bag and retuning the fluid to the infusion bag via an output element.

34. The method according to claim 33, wherein the output element is a hollow and sharp needle that is arranged to puncture the infusion bag.

35. The method according to claim 22, wherein at least one of the input tube and the output tube comprises a mixture of a metal powder and at least one other material.

36. The method according to claim 35, wherein the metal powder is placed such as not to contact an organ of the patient once inserted in the nose upper canal of the patient.

37. The method according to claim 35, wherein the at least one tube comprises an inner lumen that comprises the metal powder and an outer lumen that surrounds the inner lumen.

38. The method to claim 22, wherein at least one of the input tube and the output tube comprises a mixture of particles of a heat conducting material and of another material.

39. The method according to claim 22, comprising controlling the temperature control and fluid supply unit based on a desired fluid temperature and a temperature measured by the temperature sensor.

40. The method according to claim 29, comprising sensing the temperature of the fluid by a temperature sensor that is arranged to contact the fluid.

41. The method according to claim 29, comprising sensing the temperature of the fluid by a temperature sensor without contacting the fluid.

42. The method according to claim 29, comprising sensing, by the temperature sensor, a temperature of an organ of a patient once the temperature sensor is inserted in the nose upper canal of the patient.

43. The method according to claim 22, comprising controlling power supplied to the thermoelectric heat pump.

44. The method according to claim 22, comprising dissipating heat from the thermoelectric pump by a fan and a heat sink.

45. The method according to claim 22, wherein an overall weight of the system ranges between 1 and 5 kg.
